(19) United States
(12) Patent Application Publication
(10) Pub. No.: US 2009/0039100 A1
(43) Pub. Date: Feb. 12, 2009

(54) Hand-held bandage dispenser

(76) Inventor: James King, East Bronx, NY (US)

Correspondence Address: JAMES RAY & ASSOCIATES, 2640 PITCAIRN ROAD, MONROEVILLE, PA, 15146, US

(21) Appl. No.: 12/221,830

(22) Filed: Aug. 7, 2008

Related U.S. Application Data
(60) Provisional application No. 60/955,189, filed on Aug. 10, 2007.

(51) Int. Cl. A61F 15/00

(52) U.S. Cl. 221/88

(57) ABSTRACT

A portable hand-held bandage dispenser includes a cartridge containing a multiplicity of self-adhesive bandages, and a dispenser with an end slot housing the bandages. In addition, a feeding mechanism is disposed within and outside the dispenser housing for advancing the uppermost self-adhesive bandage into contact with an ejection device. The ejection device engages a top surface of the bandages and pushes it at least partially to the exterior of the dispenser housing. A spring disposed within the dispenser housing returns the feeding mechanism to a neutral position.

### CROSS REFERENCE TO RELATED APPLICATION

This patent application is related to and claims priority from U.S. Provisional Patent Application Ser. No. 60/955,189 filed Aug. 10, 2007.

### FIELD OF THE INVENTION

The present invention relates, in general, to devices for supplying small medical bandages and, more particularly, this invention relates to portable hand-held dispensers for storing multiple sterile bandages and ejecting them one at a time as needed.

### BACKGROUND OF THE INVENTION

Prior to the conception and development of the present invention, small plastic adhesive bandage carriers were readily available for a small number of bandages with manual removal from the dispenser and peeling back of the protecting film. Bulky wall or countertop dispensers have been disclosed, such as by Garland in U.S. Pat. No. 5,065,894, and Roy in U.S. Pat. No. 6,662,967. Ko in U.S. Pat. No. 5,271,522 and Fienup et al in U.S. Patent Application Publication No. 2005/0258183 disclose bandage dispensers that pull the next bandage into position as one is manually pulled out.

Damikolas in U.S. Pat. No. 6,213,343 and Kimbrell in U.S. Pat. No. 6,299,018 both disclose portable bandage dispensers that rely on a roll of bandages inside the device. The drawback to this is the need for a specialty roll of bandages to load the dispenser. Also, the drive mechanisms appear to be rather involved and are likely to be relatively expensive. The Damikolas device also strips off the liner as it dispenses adhesive bandages.

### SUMMARY OF THE INVENTION

The present invention provides a portable hand-held bandage dispenser including a cartridge containing a multiplicity of self-adhesive bandages, and the dispenser housing cartridge further having an end slot with a movable gate. In addition, a feeding mechanism is disposed within and outside the dispenser housing for advancing the uppermost self-adhesive bandage into contact with an ejection device. The ejection device engages a top surface of the bandages and pushes it at least partially to the exterior of the dispenser housing where it can be applied to an injured person. A spring disposed within the dispenser housing returns the feeding mechanism to a neutral position.

### OBJECTS OF THE INVENTION

It is, therefore, one of the primary objects of the present invention to provide a convenient hand-held device that holds and rapidly dispenses sealed bandages as needed.

Another object of the present invention is to provide a simple economical bandage dispenser that can be of a disposable type.

Still another object of the present invention is to provide a simple hand-held bandage dispenser that can be refillable with standard retail adhesive bandages.

Yet another object of the present invention is to provide a sterile adhesive bandage to an injured person with minimal human contact from the provider.

An additional object of the present invention is to provide a dispenser that can eject bandages that make it easier to remove the protective film.

In addition to the various objects and advantages of the present invention described with some degree of specificity above, it should be obvious that additional objects and advantages of the present invention will become more readily apparent to those persons who are skilled in the relevant art from the following more detailed description of the invention, particularly, when such description is taken in conjunction with the attached drawing figures and with the appended claims.

### BRIEF DESCRIPTION OF THE DRAWINGS

FIG. 1 is a side elevation view of the present invention gripped ready for use.

FIG. 2 is a side elevation view of the present invention dispensing a self adhesive bandage.

FIG. 3 is an end elevation detail view.

FIG. 4 is a side elevation sectional view.

FIG. 5 is a side elevation sectional view of a preferred embodiment of the present invention.

### DETAILED DESCRIPTION OF A PRESENTLY PREFERRED AND VARIOUS ALTERNATIVE EMBODIMENTS OF THE INVENTION

Prior to proceeding to the more detailed description of the present invention it should be noted that, for the sake of clarity and understanding, identical components which have identical functions have been identified with identical reference numerals throughout the several views illustrated in the drawing figures.

Referring initially to FIG. 1, the bandage dispenser 12 is sized and contoured to fit in a person's hand with a lift trigger 16 near the heel of the hand and the thumb adjacent a push lever 18. An internal cartridge 14 holds a stack of adhesive bandages. A leaf spring 15 keeps the lift trigger 16 extended from the dispenser 12 when in the neutral position.

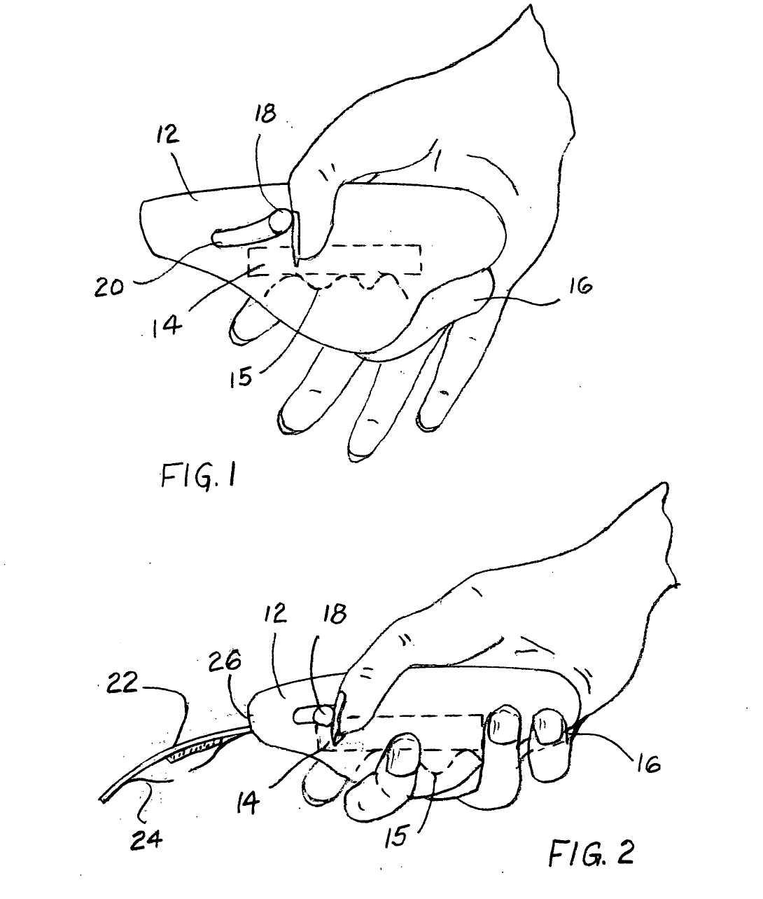

FIG. 2 illustrates the bandage dispenser 12 in action. The lift trigger 16 is squeezed up with the fingers thereby pushing on the leaf spring 15 which in turn raises the bandages in the cartridge 14. The push lever 18 is then forced by the thumb along the track 20 such as to skim the top bandage 22 off the top and out the end slot 26. While the unit will operate with conventional adhesive bandages, further sterile advantage may be realized if the protective peelable film 24 on the bandages is split closer to the end ejected first. With the adhesive covering film 24 slit near the outer end, it should then be possible, with little or no touching by the provider, to pull off film 24 and get the bandage 22 to stick on the target area of skin and subsequently pull back on dispenser 12 leaving the bandage on the skin. The levers 16 and 18 can then be released until the next time a bandage is needed.

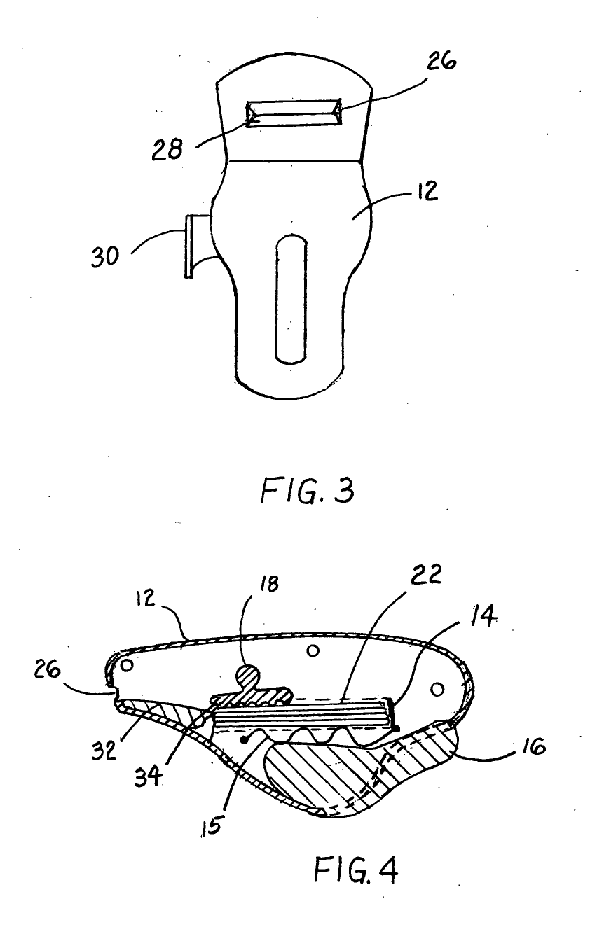

FIG. 3 provides end-view detail of the dispenser 12. The slot 26 can optionally have a lightweight flexible gate 28 that keeps dust and dirt out, but allows the bandages to be pushed through. Also shown is an optional side clip 30 that would permit hanging it on the pocket of a lab coat or nurse's uniform.

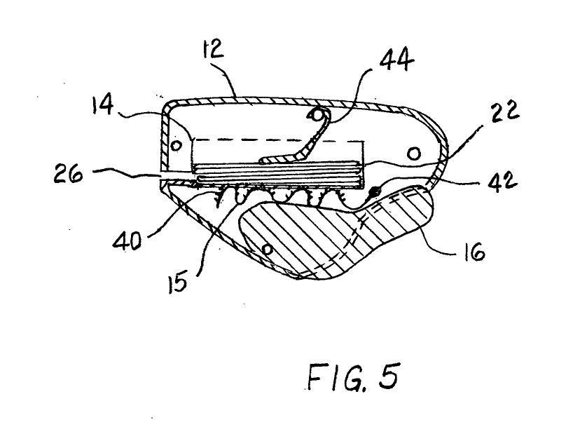

FIG. 4 is a side elevation sectional view showing some internal details of the bandage dispenser. Inside the dispenser housing 12 is a stack of bandages 22 in a cartridge 14. A lift trigger 16 protrudes from the bottom side and is in contact with a leaf spring 15. The opposite side of the leaf spring 15 extends through a bottom slot in the cartridge 14 and contacts an optional loose movable strip beneath the stack of bandages 22. When the lift trigger 16 is pushed up, it transmits upward force to the leaf spring 15 which in turn lifts the stack of bandages 22. While the stack is raised, the slide lever 18 is pushed forward, and the teeth on the pusher 34 engage the top bandage 22 and slide it along internal ramp 32. Additional pushing forces the adhesive bandage 22 out the front slot 26.

FIG. 5 is an elevation cross sectional view of the present invention in a preferred embodiment. Inside the dispenser housing 12 is a stack of bandages 22 in a cartridge 14. A lift trigger 16 protrudes from the bottom side and is in contact with a sinusoidal leaf spring 15. The opposite side of the leaf spring 15 extends partially through a bottom slot in the cartridge 14 and has teeth 40 in contact with at least half of the lowest bandage in the stack of bandages 22. A top-side leaf spring 44 maintains downward pressure on the stack of bandages 22 as the stack of bandages grows shorter. The leaf spring 15 is anchored at only one end 42. When the lift trigger 16 is pushed up, it transmits upward and forward force to the sinusoidal leaf spring 15 which in turn tends to flatten the sinusoidal leaf spring 15 and lengthen it with the aid of force from the top-side leaf spring 44. The teeth 40 on the top side of leaf spring 15 grasp the one side of the bottom bandage in the stack and push it out the chute 26. The end of a bandage 22 will extend out the chute 26 far enough to be grabbed manually or stuck onto a subjects skin once some of the protective film is peeled off. This embodiment may not eject the bandage as far as the aforementioned design will, but it offers the advantage of requiring just a single action on the part of the user.

While a presently preferred embodiment of the present invention has been described in sufficient detail above to enable a person skilled in the relevant art to make and use the same, it should be obvious that various other adaptations and modifications can be envisioned by those persons skilled in such art without departing from either the spirit of the invention or the scope of the appended claims.

I claim:

1. A portable hand-held bandage dispenser comprising:
a) a cartridge for containing a multiplicity of self-adhesive bandages;
b) a dispenser housing with at least one hollow zone for storing and transporting said cartridge, said housing further including an end slot;
c) an ejection means disposed within said dispenser housing and having an exterior operator for engaging a top surface of uppermost of said self-adhesive bandages and ejecting said uppermost bandage at least partially to exterior of said dispenser housing; and
d) a feeding means disposed partially within and partially outside said dispenser housing for advancing said uppermost of said self-adhesive bandages into contact with said ejection means.

2. The portable handheld bandage dispenser, according to claim 1, wherein said ejection means is a finger activated lever in a slotted track.

3. The portable handheld bandage dispenser, according to claim 1, wherein said feeding means is a finger-activated trigger.

4. The portable handheld bandage dispenser, according to claim 1, wherein said feeding means further includes a spring means disposed within said dispenser housing for returning said feeding means to a neutral position.

5. The portable handheld bandage dispenser, according to claim 1, wherein said ejection means includes an internal saw-toothed surface attached to said external operator.

6. The portable handheld bandage dispenser, according to claim 1, wherein said end slot further includes a flexible gate.

7. A portable hand-held bandage dispenser comprising:
a) a cartridge for containing a multiplicity of self-adhesive bandages;
b) a dispenser housing with at least one hollow zone for storing and transporting said cartridge, said housing further including an end chute;
c) an ejection means disposed within said dispenser housing and having a partially exterior operator for engaging a surface of lowermost of said self-adhesive bandages and ejecting said lowermost bandage at least partially to exterior of said dispenser housing; and
d) an internal leaf spring disposed with one end anchored inside said dispenser housing and an opposite end applying downward pressure to said multiplicity of self-adhesive bandages.

8. The portable handheld bandage dispenser, according to claim 7, wherein said ejection means is a sinusoidal leaf spring in contact with said partially exterior operator which is a finger-activated trigger.
